(19) United States
(12) Patent Application Publication
(10) Pub. No.: US 2020/0276455 A1
(43) Pub. Date: Sep. 3, 2020

(54) APPARATUS FOR PRECISE POSITIONING OF BRACHYTHERAPY TEMPLATE

(71) Applicant: Breast Microseed, Inc., Mercer Island, WA (US)

(72) Inventors: Emily Lin, Mercer Island, WA (US); Christopher Porter, Mercer Island, WA (US); Kevin Kelley, Mercer Island, WA (US); Steve Boni, Mercer Island, WA (US); Byron Stuck, Mercer Island, WA (US)

(21) Appl. No.: 16/801,102

(22) Filed: Feb. 25, 2020

Related U.S. Application Data
(60) Provisional application No. 62/812,193, filed on Feb. 28, 2019.
(60) Provisional application No. 62/877,772, filed on Jul. 23, 2019.

(51) Int. Cl. A61N 5/10

(52) U.S. Cl. CPC A61N 5/1049; A61N 5/1007; A61N 5/1065; A61N 5/1081; A61N 2005/1051; A61N 2005/1063; A61N 2005/1012; A61N 2005/1009; A61N 5/1016

(57) ABSTRACT

An apparatus for precise positioning of a template and method for precise positioning of the template may be used in a brachytherapy procedure. Several different embodiments of the precise positioning apparatus may be used.

### RELATED APPLICATIONS

This application claims the benefit of U.S. Provisional Application No. 62/812,193, filed Feb. 28, 2019 and U.S. Provisional Application No. 62/877,772, filed Jul. 23, 2019, both of which are incorporated herein by reference.

### FIELD

The disclosure relates generally to a mechanism and method for radiation oncology.

### BACKGROUND

When women are treated for breast cancer (which is the most commonly diagnosed cancer in women), they can opt for a mastectomy (complete removal of the breast tissue) or a breast conservation surgery. Due to the use of widespread screening mammograms, women are diagnosed with localized and early-stage disease so that the breast conservation surgery followed by radiation treatment may be used. The typical radiation treatment is adjuvant breast radiation. While the adjuvant breast radiation results in good survival rates, adjuvant breast radiation treatment typically takes 3.5 to 7 weeks which is too long. In addition, since the adjuvant breast radiation treatment is typically provided using external beam radiation, there is a greater risk of acute skin reactions due to the healthy tissue interaction with the radiation.

As a result, accelerated partial breast irradiation may be used which results in a quicker treatment time and less radiation-induced acute skin reactions. One technique used for the accelerated partial breast irradiation is brachytherapy. In one method, radioactive sources are permanently implanted into the breast tissue at the site of the surgery wherein the radioactive sources may be high dose or low dose.

Currently there are a handful of ways to insert radioactive sources into breast tissue. One is by a free hand method, another uses a compressive template device to temporary hold insertion catheters and the last uses a locking template system and non-fixated fiducial needle. These methods are limited in that they do not ensure the sources are placed in the desired location as prescribed by the treatment plan 100% of the time. The lack of ability to place the radioactive sources in the desired location means that the remaining tumor margin is not receiving the appropriate radiation and healthy tissue is receiving unwanted radiation.

In the high dose rate brachytherapy area, a clinician would place hollow catheters into the breast to facilitate the insertion of a temporary radioactive source per a treatment plan which are then removed once the treatment is completed. The placement of these catheters may be by either free hand directly into the breast or by free hand though compressive template systems used to stereo-tactically immobilize the breast. Both Varian Medical Systems and Nucletron offer commercially available template immobilization products.

In the low dose rate brachytherapy area, one method for permanent breast radioactive seed implantation is described in detail in “First Report of a Permanent Breast103PD Seed Implant As Adjuvant Radiation Treatment for Early-Stage Breast Cancer”, Dr. Jean-Philippe Pignol et al., International Journal of Radiation Oncology Biological Physics, Vol. 64, No. 1, pp. 176-181 (2006) which is incorporated herein by reference. This method uses a non-fixated fiducial needle, locking template and stereotactic fixation to insert lose dose rate (LDR) radioactive source strands into the treatment site. In this method, the fiducial needle can migrate/move once inserted thus changing the depth at which the source strands are deployed. In addition, the system is very cumbersome to use and is not user intuitive. The user manually operates the locking template and stereotactic fixation system by turning knobs to adjust and lock/unlock positions. The position and orientation of the locking template is determined by a treatment planning software. The correct position and orientation of the template are verified by other instruments such as an inclinometer and a ruler. Overall, the system can be difficult to use in a reproducible and precise manner.

Thus, it is desirable to provide an apparatus that can position and immobilize the template with the goal of improving the reproducibility of the procedure and ensuring that the sources are reliably and consistently inserted in an exact position per a patient prescription treatment plan from patient to patient as well as improve the ease-of-use of the device and procedure. It is to this end that the disclosure is directed.

### BRIEF DESCRIPTION OF THE DRAWINGS

FIG. 1 illustrates an example of a first embodiment of an apparatus for positioning a template;

FIG. 2 illustrates a method of positioning a template using the apparatus in FIG. 1,

FIG. 3 illustrates an example of a second embodiment of an apparatus for positioning a template;

FIG. 4 illustrates a method for positioning the template using the apparatus in FIG. 3;

FIG. 5 illustrates an example of a third embodiment of an apparatus for positioning a template;

FIG. 6 illustrates a method for positioning the template using the apparatus in FIG. 5;

FIG. 7 illustrates an example of a fourth embodiment of an apparatus for positioning a template; and

FIGS. 8A and 8B illustrate a method for implanting radioactive sources into a patient with precise positioning of brachytherapy template.

### DETAILED DESCRIPTION OF ONE OR MORE EMBODIMENTS

It is to be understood that the present invention is not limited to the embodiment(s) described above and illustrated herein, but encompasses any and all variations falling within the scope of any claims. For example, references to the present invention herein are not intended to limit the scope of any claim or claim term, but instead merely make reference to one or more features that may be covered by one or more of the claims. Materials, processes and numerical examples described above are exemplary only, and should not be deemed to limit the claims. Further, as is apparent from the claims and specification, not all method steps need be performed in the exact order illustrated or claimed, but rather in any order.

It is noted that the following disclosure is particularly applicable to radioactive source implantation into breast tissue and it is in this context that the disclosure will be described. It will be appreciated, however, that the device and method have greater utility since the device can be used with other devices besides the template, needles, and radioactive sources and in various different tissues. Furthermore, the apparatus described can be used for any brachytherapy process to position a template and is not limited to radioactive source implantation into breast tissue and the apparatus may be used in a medical treatment of various diseases and is not limited to breast cancer.

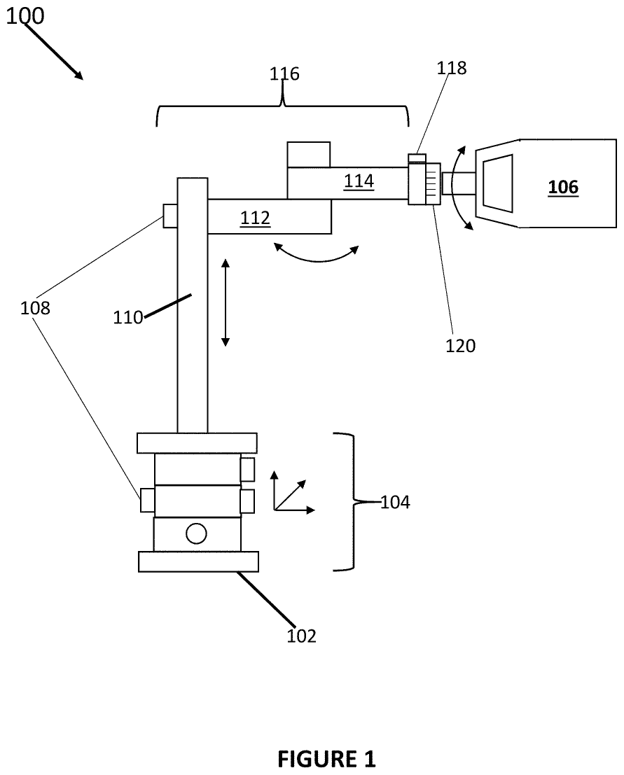

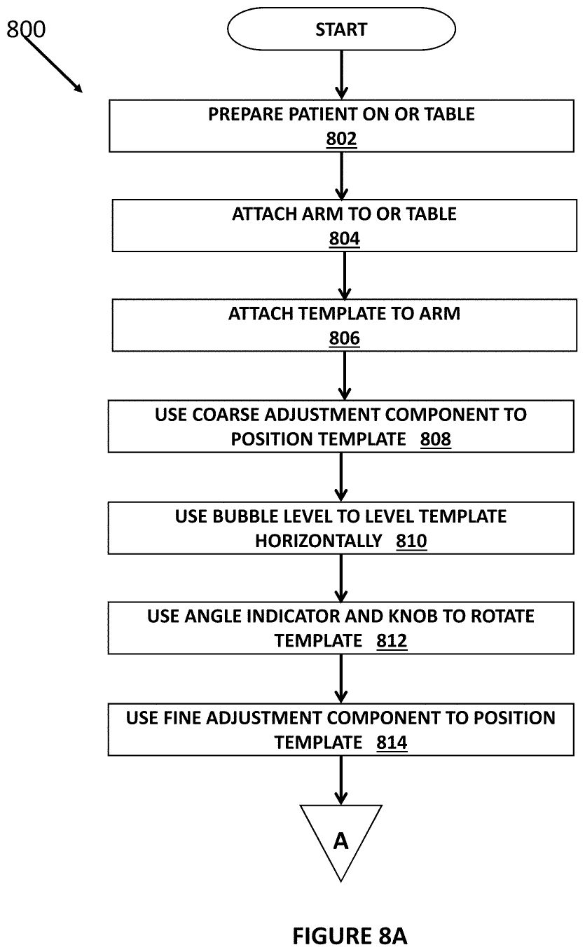

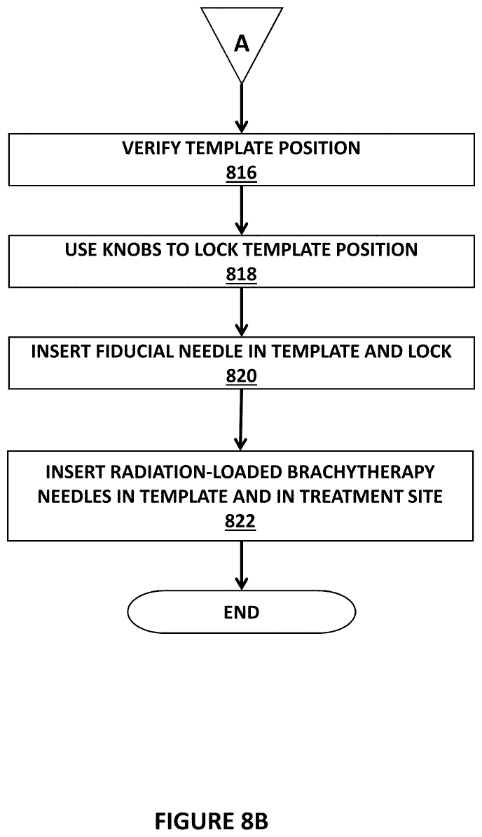

FIG. 1 illustrates an example of a first embodiment of an apparatus 100 for positioning a template, such as a template used in a brachytherapy process to implant radioactive sources into a patient as shown in FIGS. 8A and 8B and described below. The apparatus 100 may be fixed to a surface, such as a patient bed/operating table, a railing of the operating table and the like, using a known attachment mechanism 102 and may have a fine adjustment component 104 that permits fine adjustment in three axes of freedom of a template 106 position relative to a patient or treatment zone/area. The template 106 may be made of various materials and may have an array of holes in the template that may be used to accurately position one or more fiducial needles and one or more radioactive source needles into a treatment site, such as breast tissue, to position the radioactive sources at the treatment site. In some embodiments, the template may have one or more fiducial needles holes that can lock the fiducial needle once the fiducial needle is positioned and one or more radioactive source needle holes that do not lock the radioactive source needles.

In the embodiment shown in FIG. 1, the fine adjustment component 104 may be located adjacent to the attachment mechanism 102, but may also be in other locations. The fine adjustment component may have positioning knobs to adjust the position of the template linearly or rotationally. The mechanism of movement in the fine adjustment component may be mechanical (for example, rack and pinion, lead screw, ball screw, etc.), motorized, or a combination of mechanical and motorized mechanisms. The fine adjustment component 104 may have a locking knob or a plurality of locking knobs (one for each type of movement, linearly or rotationally). 108 that may be turned to prevent further adjustments once the proper adjustment is achieved. In general, the final adjustments may be performed towards the end of the method for positioning the template adjacent the treatment site.

The apparatus 100 may further comprise an arm 110 that may move vertically relative to the surface on which the apparatus 100 is fixed and thus adjust the template 106 position vertically. The apparatus 100 may also have an upper arm 112 (located on an opposite end of the arm 110 from the fine adjustment component 104) that is attached to the arm 110 and permits horizontal positioning relative to the surface on which the apparatus 100 is fixed of the template 106. The upper arm 112 may also have a locking knob 108 that may be turned to prevent further adjustments once the proper adjustment is achieved. The upper arm 112 and a second horizontal arm 114 and locking knob 108 may together be a course adjustment component 116 for larger (and thus more coarse) adjustment of the position of the template 106. The course adjustment component 116 moves along multiple axes (linearly and/or rotationally) and the course adjustment component 116 may include, for example, an articulated arm in which the template moves along one or multiple planes via the rotation of at least one joint, a rack and pinion movement in a mechanical or power-assisted manner in which the template moves linearly or along one plane at a time, a leadscrew-driven movement in a mechanical or power-assisted manner in which the template moves linearly or along one plane at a time and/or a motorized or robotic arm in which the template moves linearly and/or rotationally.

An end of the course adjustment component 114 adjacent to the template 106 may have a leveling device 118, such as a bubble level device for example, and an angle indicator and knob 120 that adjusts the rotational movement of the template and permit the user to verify that the template is level (horizontally or vertically or both) using the bubble level 118. The level 188 may be implemented, for example, as one or more bubble levels placed on the appropriate axis of the apparatus and/or sensors placed on the appropriate axis of the apparatus that measure whether the template is level along a particular axis. The angle indicator and knob 120 permit the angle of the template 106 relative to the treatment site to be precisely adjusted and then locked using the knob. The angle indicator and knob 120 may be implemented to permit mechanical rotation of the template and/or motorized or automatic rotation of the template. Using this apparatus, the position of the template may be precisely positioned. Furthermore, the template may be precisely positioned during a treatment in which radioactive sources are placed into tissue, such as the breast.

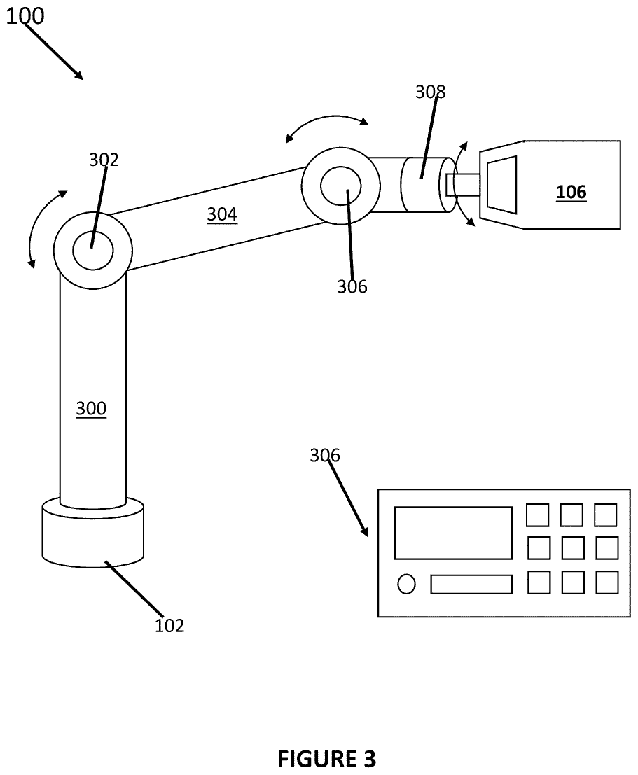

The apparatus 100 may also have an element that can determine one or more angles (e.g., pitch, roll, yaw) of the brachytherapy template 106. The angle determining element may be, for example, a mechanical angle indicator and/or known sensors that detect changes in orientation and converts the input into a digital readout of the angle. The apparatus 100 may be controlled manually as shown in FIG. 1 and/or electronically as shown in FIG. 3. During the manual control, a user manually turns a knob to move a component of the apparatus one way or another. In the electronic embodiment, a program input via interface or software—e.g., input coordinates that move the brachytherapy template to a preselected position that may be driven, for example, by the treatment plan for a particular patient. In the electronic embodiment, each of the elements of the apparatus 100 shown in FIG. 1 may have a motor or other device that can be controlled by control signals from a computer.

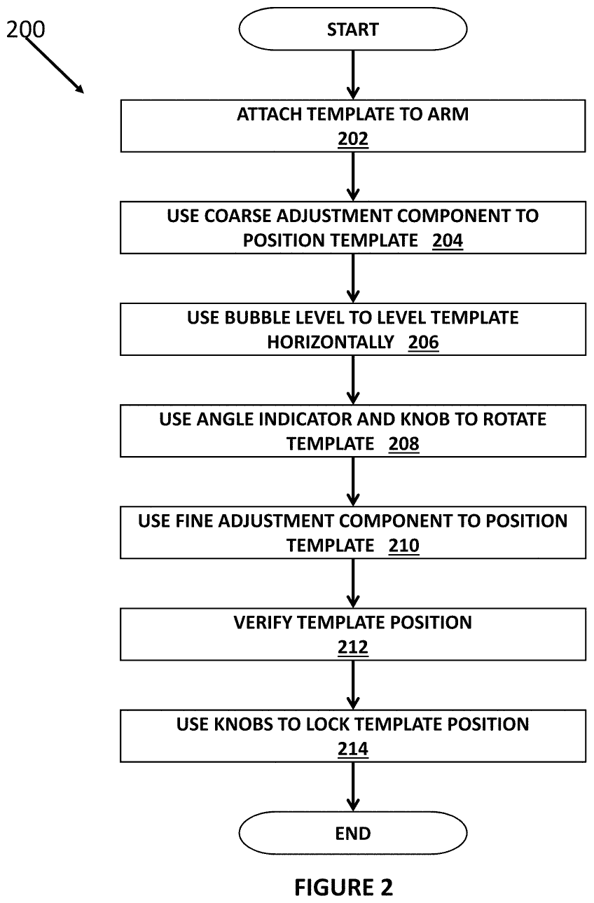

FIG. 2 illustrates a method 200 of positioning a template using the apparatus in FIG. 1. In the method, the template 106 may be attached to the apparatus 100 (202) and the coarse adjustment component 116 may be used to position the template 106 initially (204). The bubble level 118 may be used level the template horizontally (206). The angle indicator and knob 120 may be used to rotate the template (208). Then, the fine adjustment component 104 may be used to position the template (210) into its final treatment position. A person that is positioned the template 106 may verify the positioning (212) and then use the knobs 108 to lock the template position (214). The coarse adjustment of the template allows the template to move within a space that is 40 inches long, 16 inches high, and 9 inches deep; though, it is noted that the range of coarse adjustment is dependent on the mechanism. The fine adjustment of the template allows the template to move incrementally within a 2 inch range (up/down, left/right, in/out); though, it is noted that the range of the fine adjustment is dependent on the mechanism. The positioning of the template can be verified with tools such as an inclinometer, ruler, or laser height gauge. These tools can be internally built into the apparatus or used externally. It is noted that the apparatus can run a software that verifies the positioning the template using electronic components such as sensors, chips, and etc.

Once the template in positioned as shown in FIG. 2, a brachytherapy treatment can be performed in which, using the positioned template: 1) one or more fiducial needles may be positioned into the tissue of a patient and locked to the template; 2) one or more radioactive source needles may be guided into the tissue using the template; 3) the one or more radioactive sources may be positioned (based on a treatment plan and the dose profile of each radioactive source) in the tissue to treat the tissue with the various needles being withdrawn from the tissue leaving the radioactive sources positioned in the tissue. In one embodiment, the tissue may be breast tissue and the radioactive sources may be Iodine-125, Palladium-103, etc.

FIG. 3 illustrates an example of a second embodiment of an apparatus 100 for positioning a template. The apparatus has the same attachment mechanism 102 that attaches to a patent operating table and further has a first vertical arm 300 (shown in the example in FIG. 3 as having a circular cross section) that is connected by a first joint 302 to a second arm 304 wherein the joint 302 allows the position and angle of the second arm 304 relative to the first arm 300 to be adjusted. The apparatus 100 may further have a third joint 306 at an end of the second arm opposite of the second joint that is connected to a third arm 308 that in turn can be coupled to the template 106. The joint 306 allows the position and angle of the third arm 308 to be adjusted relative to the second arm 304. The third arm 308 may allow a rotational motion of the template 106 as shown. Each of the elements 300-308 that may motorized and may be controlled by a controller 306 that generates and issued commands/signals to each element to precisely position the template. The controller 306 may include a processor and memory and a plurality of lines of instructions/computer code that generate the commands for each element based on user input (not user input devices on controller 306) and/or based on the previously determined treatment plan for each particular patient.

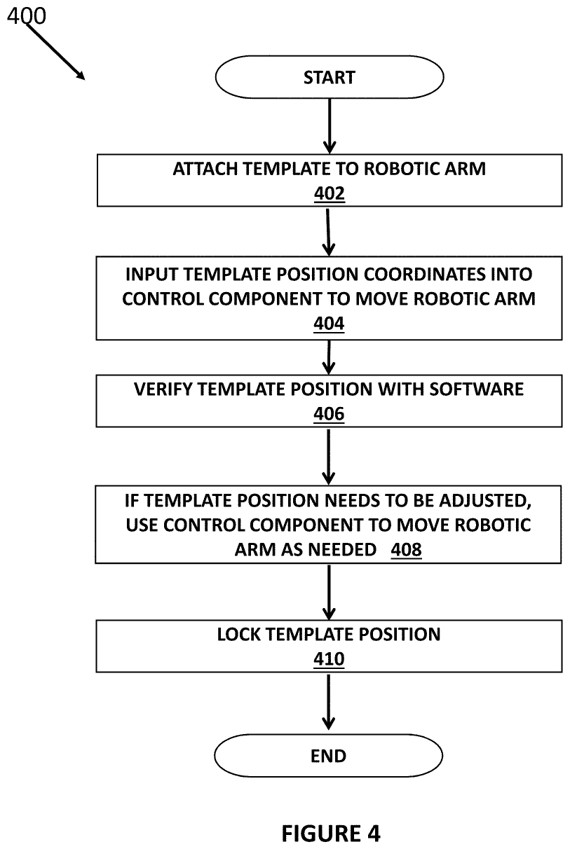

FIG. 4 illustrates a method 400 for positioning the template using the apparatus in FIG. 3. The method 400 may also be performed by a different apparatus that permits the position of the template 106 to be controlled by a computer and motors. In the method, the template 106 may be attached to the apparatus 100 (402). In this method in which the position of the template is controlled by a computer and motors, an operator of the apparatus 100 may input template position coordinates (for the particular patient based on the treatment plan for the particular patient) into the control component (such as the controller 306 in FIG. 3 for example) (404) to move the robotic arm(s) of the apparatus. The controller (and the software in the controller) may be used to verify the correct template position (406) and then the controller uses the elements of the apparatus (generates commands for one or more elements) to adjust the template position (408). Once the template is properly positioned, the template may be locked in position (410) either manually or programmatically by the controller. As with other methods, once the template is locking into position, the brachytherapy treatment as described in other embodiments may be performed.

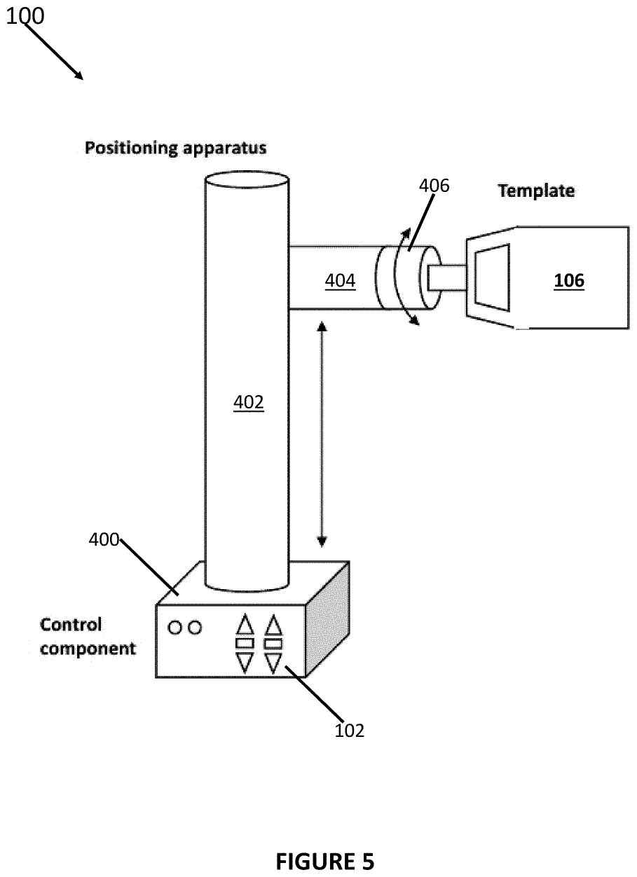

FIG. 5 illustrates an example of a third embodiment of an apparatus 100 for positioning a template 106. In this embodiment, the apparatus 100 has the same attachment mechanism 102 for attachment to the operating table that has an integrated controller 400 as shown that has the same characteristics and elements of the controller 306 shown in FIG. 3. The apparatus 100 may have a positioning apparatus that includes a first arm 402 and a second arm 404 in which the second arm 404 may be programmatically moved vertically relative to the first arm 402. An end of the second arm 404 may have a third rotating element 406 that allows the template 106 to be rotated relative to the first and second arms 402, 404. This embodiment, like the embodiment in FIG. 3, allows the positioning of the template 106 to be either manually controlled and/or programmatically controlled by the controller 400.

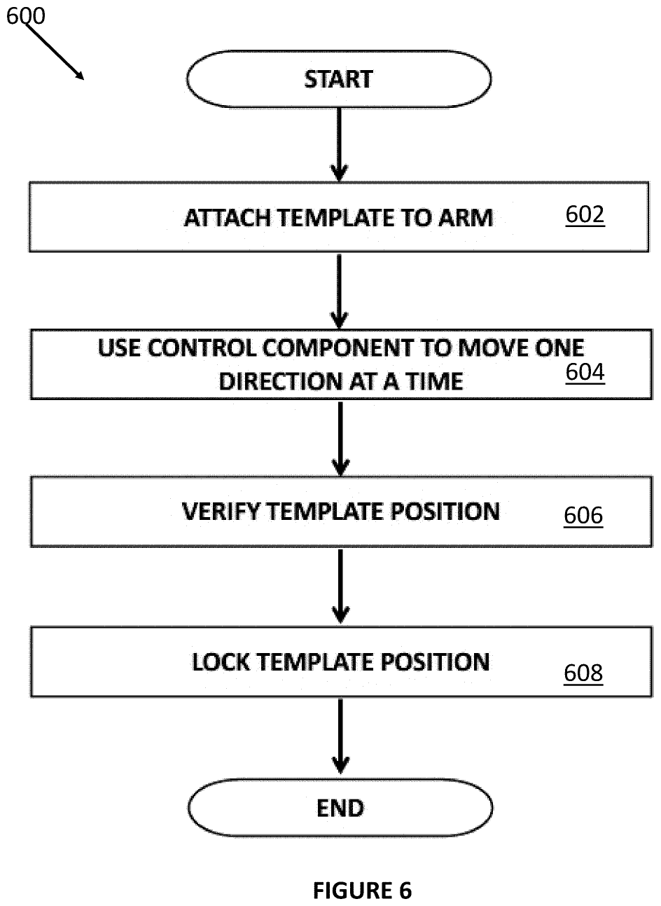

FIG. 6 illustrates a method 600 for positioning the template using the apparatus in FIG. 5. Like the method in FIG. 4, the method may be performed by other apparatus that can position a template and the template 106 may be attached to the apparatus (602). In the method, the controller/control component may be used to move the apparatus and template 106 in one direction at a time (604) until the correct position of the template is achieved and the template position is verified (606). Once the template is properly positioned, the template may be locked in position (608) either manually or programmatically by the controller. As with other methods, once the template is locking into position, the brachytherapy treatment as described in other embodiments may be performed.

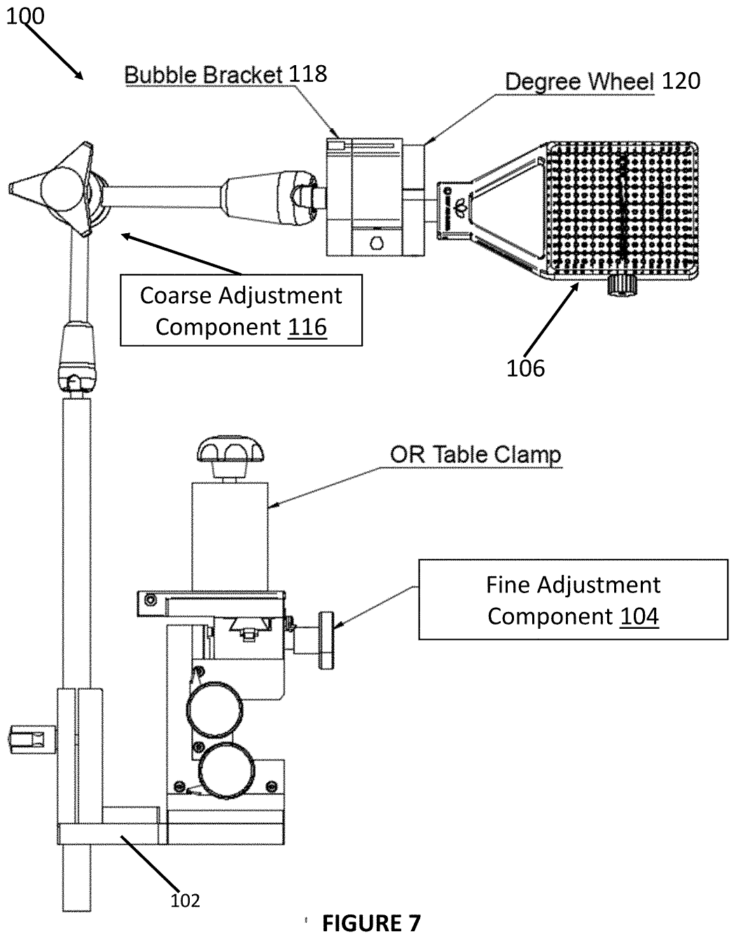

FIG. 7 illustrates an example of a fourth embodiment of an apparatus 100 for positioning a template 106 for a brachytherapy treatment. The fourth embodiment shows the apparatus 100 implements using currently commercially available medical elements including the OR table fixation device, such as a clamp, and the other known elements including the arms of the apparatus and the locking joints that are all commercially sold and commonly used for patient arm positioning, anesthetic/breathing tubes, surgical instrument holders, and etc. Added to those known elements are the bubble bracket 118 as described above and the degree wheel 120. The bubble levels on the bubble bracket 118 are typically used to calibrate medical equipment.

FIGS. 8A and 8B illustrate a method to place one or more radioactive seeds into a treatment region of the patient using the precise template positioning device and methods described above. In one example, the brachytherapy method may be used to place radioactive seeds into breast tissue on a patient who has breast cancer and may or may not have had a lumpectomy to remove the tumor. In the method, a patient on an operating room table (802) is prepared for the procedure. A precise positioning apparatus as described above may be attached to the operating room table (804) and then the brachytherapy template may be attached to the precise positioning apparatus (806). The coarse adjustment component 116 of the precise positioning apparatus may be used (808) to position the brachytherapy template 106 initially. The bubble level 118 may be used to level the template horizontally (810) with respect to the operating room table and the patient. The angle indicator and knob 120 may be used to rotate the template (812) relative to the precise positioning device. Then, the fine adjustment component 104 may be used to position the template (814) into its final treatment position. The coarse adjustment of the template allows the template to move within a space that is 40 inches long, 16 inches high, and 9 inches deep; though, it is noted that the range of coarse adjustment is dependent on the mechanism. The fine adjustment of the template allows the template to move incrementally within a 2 inch range (up/down, left/right, in/out); though, it is noted that the range of the fine adjustment is dependent on the mechanism. The positioning of the template can be verified with tools such as an inclinometer, ruler, or laser height gauge. These tools can be internally built into the apparatus or used externally. It is noted that the apparatus can run a software that verifies the positioning the template using electronic components such as sensors, chips, and etc.

A person that is positioning the template 106 may verify the positioning (816) and then use the knobs 108 to lock the template position (818) in order to perform the placement of the one or more radioactive seeds into the tissue of the patient, such as the breast tissue of the patient. The position of the template may be verified by various techniques including, for example, software analysis that compares the position of the template with the treatment plan and/or sensors placed at or near the target site act as reference point for the apparatus. The locking of the template may be accomplished by various techniques including, for example, mechanical locking—turning a knob or screw and/or automated locking when correct position of the brachytherapy template is verified with the software. The automated locking can be performed when the apparatus is controlled manually or electronically.

Once the template in positioned, a brachytherapy treatment can be performed in which, using the positioned template: 1) one or more fiducial needles may be positioned into the tissue of a patient and locked to the template (820); 2) one or more radioactive source needles may be guided into the tissue using the template; 3) the one or more radioactive sources may be positioned (822) (based on a treatment plan and the dose profile of each radioactive source) in the tissue to treat the tissue with the various needles being withdrawn from the tissue leaving the radioactive sources positioned in the tissue. In one embodiment, the tissue may be breast tissue and the radioactive sources may be Iodine-125, Palladium-103, etc.

The foregoing description, for purpose of explanation, has been described with reference to specific embodiments. However, the illustrative discussions above are not intended to be exhaustive or to limit the disclosure to the precise forms disclosed. Many modifications and variations are possible in view of the above teachings. The embodiments were chosen and described in order to best explain the principles of the disclosure and its practical applications, to thereby enable others skilled in the art to best utilize the disclosure and various embodiments with various modifications as are suited to the particular use contemplated.

The system and method disclosed herein may be implemented via one or more components, systems, servers, appliances, other subcomponents, or distributed between such elements. When implemented as a system, such systems may include an/or involve, inter alia, components such as software modules, general-purpose CPU, RAM, etc. found in general-purpose computers. In implementations where the innovations reside on a server, such a server may include or involve components such as CPU, RAM, etc., such as those found in general-purpose computers.

Additionally, the system and method herein may be achieved via implementations with disparate or entirely different software, hardware and/or firmware components, beyond that set forth above. With regard to such other components (e.g., software, processing components, etc.) and/or computer-readable media associated with or embodying the present inventions, for example, aspects of the innovations herein may be implemented consistent with numerous general purpose or special purpose computing systems or configurations. Various exemplary computing systems, environments, and/or configurations that may be suitable for use with the innovations herein may include, but are not limited to: software or other components within or embodied on personal computers, servers or server computing devices such as routing/connectivity components, hand-held or laptop devices, multiprocessor systems, microprocessor-based systems, set top boxes, consumer electronic devices, network PCs, other existing computer platforms, distributed computing environments that include one or more of the above systems or devices, etc.

In some instances, aspects of the system and method may be achieved via or performed by logic and/or logic instructions including program modules, executed in association with such components or circuitry, for example. In general, program modules may include routines, programs, objects, components, data structures, etc. that perform particular tasks or implement particular instructions herein. The inventions may also be practiced in the context of distributed software, computer, or circuit settings where circuitry is connected via communication buses, circuitry or links. In distributed settings, control/instructions may occur from both local and remote computer storage media including memory storage devices.

The software, circuitry and components herein may also include and/or utilize one or more type of computer readable media. Computer readable media can be any available media that is resident on, associable with, or can be accessed by such circuits and/or computing components. By way of example, and not limitation, computer readable media may comprise computer storage media and communication media. Computer storage media includes volatile and nonvolatile, removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical storage, magnetic tape, magnetic disk storage or other magnetic storage devices, or any other medium which can be used to store the desired information and can accessed by computing component. Communication media may comprise computer readable instructions, data structures, program modules and/or other components. Further, communication media may include wired media such as a wired network or direct-wired connection, however no media of any such type herein includes transitory media. Combinations of the any of the above are also included within the scope of computer readable media.

In the present description, the terms component, module, device, etc. may refer to any type of logical or functional software elements, circuits, blocks and/or processes that may be implemented in a variety of ways. For example, the functions of various circuits and/or blocks can be combined with one another into any other number of modules. Each module may even be implemented as a software program stored on a tangible memory (e.g., random access memory, read only memory, CD-ROM memory, hard disk drive, etc.) to be read by a central processing unit to implement the functions of the innovations herein. Or, the modules can comprise programming instructions transmitted to a general purpose computer or to processing/graphics hardware via a transmission carrier wave. Also, the modules can be implemented as hardware logic circuitry implementing the functions encompassed by the innovations herein. Finally, the modules can be implemented using special purpose instructions (SIMD instructions), field programmable logic arrays or any mix thereof which provides the desired level performance and cost.

As disclosed herein, features consistent with the disclosure may be implemented via computer-hardware, software and/or firmware. For example, the systems and methods disclosed herein may be embodied in various forms including, for example, a data processor, such as a computer that also includes a database, digital electronic circuitry, firmware, software, or in combinations of them. Further, while some of the disclosed implementations describe specific hardware components, systems and methods consistent with the innovations herein may be implemented with any combination of hardware, software and/or firmware. Moreover, the above-noted features and other aspects and principles of the innovations herein may be implemented in various environments. Such environments and related applications may be specially constructed for performing the various routines, processes and/or operations according to the invention or they may include a general-purpose computer or computing platform selectively activated or reconfigured by code to provide the necessary functionality. The processes disclosed herein are not inherently related to any particular computer, network, architecture, environment, or other apparatus, and may be implemented by a suitable combination of hardware, software, and/or firmware. For example, various general-purpose machines may be used with programs written in accordance with teachings of the invention, or it may be more convenient to construct a specialized apparatus or system to perform the required methods and techniques.

Aspects of the method and system described herein, such as the logic, may also be implemented as functionality programmed into any of a variety of circuitry, including programmable logic devices (“PLDs”), such as field programmable gate arrays (“FPGAs”), programmable array logic (“PAL”) devices, electrically programmable logic and memory devices and standard cell-based devices, as well as application specific integrated circuits. Some other possibilities for implementing aspects include: memory devices, microcontrollers with memory (such as EEPROM), embedded microprocessors, firmware, software, etc. Furthermore, aspects may be embodied in microprocessors having software-based circuit emulation, discrete logic (sequential and combinatorial), custom devices, fuzzy (neural) logic, quantum devices, and hybrids of any of the above device types. The underlying device technologies may be provided in a variety of component types, e.g., metal-oxide semiconductor field-effect transistor (“MOSFET”) technologies like complementary metal-oxide semiconductor (“CMOS”), bipolar technologies like emitter-coupled logic (“ECL”), polymer technologies (e.g., silicon-conjugated polymer and metal-conjugated polymer-metal structures), mixed analog and digital, and so on.

It should also be noted that the various logic and/or functions disclosed herein may be enabled using any number of combinations of hardware, firmware, and/or as data and/or instructions embodied in various machine-readable or computer-readable media, in terms of their behavioral, register transfer, logic component, and/or other characteristics. Computer-readable media in which such formatted data and/or instructions may be embodied include, but are not limited to, non-volatile storage media in various forms (e.g., optical, magnetic or semiconductor storage media) though again does not include transitory media. Unless the context clearly requires otherwise, throughout the description, the words “comprise,” “comprising,” and the like are to be construed in an inclusive sense as opposed to an exclusive or exhaustive sense; that is to say, in a sense of “including, but not limited to.” Words using the singular or plural number also include the plural or singular number respectively. Additionally, the words “herein,” “hereunder,” “above,” “below,” and words of similar import refer to this application as a whole and not to any particular portions of this application. When the word “or” is used in reference to a list of two or more items, that word covers all of the following interpretations of the word: any of the items in the list, all of the items in the list and any combination of the items in the list.

Although certain presently preferred implementations of the invention have been specifically described herein, it will be apparent to those skilled in the art to which the invention pertains that variations and modifications of the various implementations shown and described herein may be made without departing from the spirit and scope of the invention. Accordingly, it is intended that the invention be limited only to the extent required by the applicable rules of law.

While the foregoing has been with reference to a particular embodiment of the disclosure, it will be appreciated by those skilled in the art that changes in this embodiment may be made without departing from the principles and spirit of the disclosure, the scope of which is defined by the appended claims.

What is claimed is:

1. An apparatus, comprising:
a precise positioning device capable of precisely positioning a template at a position, the template having a plurality of holes configured to pass one or more fiducial needles and one or more radioactive source needles through the template;
the precise positioning device further comprising:
a linear adjustment device that adjusts a horizontal position and a vertical position of the template;
a rotational adjustment device that adjusts an angular position of the template; and
a lock that locks the position of the template after it is positioned.

2. The apparatus of claim 1, wherein the linear adjustment device further comprises a coarse adjustment component configured to initially adjust the position of the template and a fine adjustment component to perform fine position adjustments of the template.

3. The apparatus of claim 2, wherein the rotational adjustment device further comprises an angle device that is capable of changing a rotational position of the template.

4. The apparatus of claim 3, wherein the lock further comprises one or more knobs that lock the position of the template.

5. The apparatus of claim 4 further comprising a leveling device configured to horizontally level the template.

6. The apparatus of claim 1, wherein the linear adjustment device further comprises an arm that changes the horizontal and vertical position of the template and a computer controller that controls the position of the arm.

7. The apparatus of claim 6, wherein the rotational adjustment device further comprises an angle device that is capable of changing a rotational position of the template.

8. The apparatus of claim 7, wherein the lock further comprises one or more knobs that lock the position of the template.

9. The apparatus of claim 8 further comprising a leveling device configured to horizontally level the template.

10. The apparatus of claim 1, wherein the linear adjustment device has a motor and the rotational adjustment device has a motor and wherein the apparatus further comprises a controller connected to the motors of the linear and rotational adjustment devices that generates control signals to each of the motors of the linear and rotational adjustment devices to move the linear and rotational adjustment devices.

11. The apparatus of claim 10, wherein the controller further comprises a processor and a treatment plan comprising a plurality of lines of instructions wherein the processor of the controller is configured to move the linear and rotational adjustment devices based on the treatment plan.

12. The apparatus of claim 1 further comprising a fixation device that secures the precise positioning device to a fixed frame of reference.

13. A method for positioning a brachytherapy template, comprising:
securing a precise positioning brachytherapy template device on a fixed frame of reference;
securing a brachytherapy template on a distal end of the precise positioning brachytherapy template device;
adjusting a vertical position and a horizontal position of the brachytherapy template using the precise positioning brachytherapy template device relative to a treatment region of a patient;
leveling the brachytherapy template relative to the treatment region of the patient using a leveling device; and
locking the brachytherapy template into a precise position related to the treatment region of the patient.

14. The method of claim 13, wherein adjusting the vertical and horizontal positions of the brachytherapy template further comprises operating a coarse adjustment component to position the brachytherapy template near the treatment region and operating a fine adjustment component to precisely position the template adjacent the treatment region.

15. The method of claim 14 further comprising operating an angle component to rotate the template at an angle relative to the treatment region of the patient.

16. The method of claim 14, wherein operating the coarse and fine adjustment components further comprises programmatically controlling the position of the coarse and fine adjustment components using a controller.

17. The method of claim 16, wherein operating the coarse and fine adjustment components further comprises generating, by the controller using a treatment plan for the patient, a command to adjust the coarse and fine adjustment components and sending the command to a motor in each of the coarse and fine adjustment components to adjust the positions of the coarse and fine adjustment components.

18. The method of claim 13, wherein securing the precise positioning brachytherapy template device on a fixed frame of reference further comprising securing the precise positioning brachytherapy template device to an operating room table on which the patient rests.

19. A method for treatment, comprising:
providing an operating table with a patient having a treatment site lying of the operating table and a precise positioning apparatus attached to the operating table;
attaching a brachytherapy template to the precise positioning apparatus;
precisely adjusting a position of the brachytherapy template relative to the treatment site using the precise positioning apparatus;
locking a position of the brachytherapy template relative to the treatment site;
inserting one or more fiducial needles, guided by medical imaging, through the template and locking the one or more fiducial needles; and
inserting one or more brachytherapy needles through the template and into the treatment site to place one or more radioactive seeds into the treatment site.

20. The method of claim 19, wherein precisely adjusting the position of the brachytherapy template further comprises operating a coarse adjustment component and a fine adjustment component to precisely position template at the treatment site.

21. The method of claim 20, wherein precisely adjusting the position of the brachytherapy template further comprises adjusting a vertical position and a horizontal position of the brachytherapy template relative to a treatment region of a patient.

22. The method of claim 21, wherein precisely adjusting the position of the brachytherapy template further comprises leveling the brachytherapy template relative to the treatment region of the patient using a leveling device.

23. The method of claim 22, wherein precisely adjusting the position of the brachytherapy template further comprises operating an angle component to rotate the template at an angle relative to the treatment region of the patient.

24. The method of claim 21, wherein adjusting a vertical position and a horizontal position of the brachytherapy template further comprises programmatically controlling the horizontal and vertical positions of the brachytherapy template using a controller.

25. The method of claim 24, wherein adjusting a vertical position and a horizontal position of the brachytherapy template further comprises generating, by the controller using a treatment plan for the patient, a command to adjust the vertical and horizontal positions of the brachytherapy template and sending the commands to a motor to adjust the vertical and horizontal positions of the brachytherapy template.
